(19) United States
(12) Patent Application Publication
(10) Pub. No.: US 2026/0337516 A1
(43) Pub. Date: Oct. 29, 2026

(54) MONO AND DIESTER LUBRICATING AGENTS FOR AQUEOUS SYSTEMS

(71) Applicant: Colonial Chemical, Inc., South Pittsburg, TN (US)

(72) Inventors: Lucas R. Moore, South Pittsburg, TN (US); Garret Bryant, Rocky Face, GA (US); Timothy G. Holtcamp, South Pittsburg, TN (US); Molly McEnery, South Pittsburg, TN (US)

(21) Appl. No.: 19/484,739

(22) PCT Filed: May 16, 2024
(86) PCT No.: PCT/US2024/029808

§ 371 Date: Nov. 14, 2025

Related U.S. Application Data
(60) Provisional application No. 63/466,957, filed on May 16, 2023.

(51) Int. Cl. C10M 173/02; A61K 8/365; A61K 8/42; A61K 8/86; A61Q 5/12; B31F 1/12; C10M 129/40; C10M 133/16; C10M 145/38; C10M 161/00; C10N 40/36

(52) U.S. Cl. CPC C10M 173/02; A61K 8/365; A61K 8/42; A61K 8/86; A61Q 5/12; B31F 1/12; C10M 129/40; C10M 133/16; C10M 145/38; C10M 161/00; C10M 2201/02; C10M 2207/126; C10M 2209/109; C10M 2215/08; C10N 2040/36

(57) ABSTRACT

A lubricity or friction reduction agent, comprising an aqueous base fluid; an emulsified PEG that is beneficial in the field of cosmetics such as hair conditioners, making cellulosic sheets and paper items as paper creping release aids, drilling mud fluids, metal working fluids, and any other applications where two substrates are in contact and a desired reduction in friction or boost in lubricity is desired.

### FIELD OF THE INVENTION

The present invention generally relates to lubricating agents. Additionally, the present invention relates to methods of improving the lubricity or friction reduction between substrates. These agents can be in formulations for the cosmetic industry such as hair conditioners. These agents can also be applied in the making of cellulosic sheets and paper items as paper creping release aids. The agents can also be applied in drilling mud fluids, metal working fluids, and any other application where two substrates are in contact and a desired reduction in friction or boost in lubricity is desired.

### BACKGROUND AND SUMMARY OF THE INVENTION

One embodiment of the present invention relates to the creping of wet-laid papers, and especially agents for reducing adhesion of paper to a hot surface of equipment.

The method involves the addition of an effective amount of the fatty acid (C₁₀₋₁₈) mono or diester of polyethylene glycol (PEG), and combinations thereof, in combination with an emulsifying agent and a carrier. In aspects of the invention, the carrier may be water.

In the manufacture of certain wet-laid paper products such as facial tissue, bathroom tissue, paper towels, filter paper, etc., the paper web is conventionally subjected to a creping process in order to give it desirable textural characteristics, such as softness and bulk. The creping process typically involves adhering the web to a rotating creping cylinder, such as the apparatus known as a Yankee dryer, and then dislodging the adhered web with a doctor blade. The impact of the web against the doctor blade ruptures some of the fiber-to-fiber bonds within the web and causes the web to wrinkle or pucker.

The severity of this creping action is dependent upon a number of factors, including the degree of adhesion between the web and the surface of the creping cylinder. Greater adhesion causes increased softness, although generally with some loss of strength. In order to increase adhesion, an adhesive creping aid is used to enhance any naturally occurring adhesion that the web may have due to its water content, which will vary widely depending on the extent to which the web has been previously dried.

Creping release aids, such as those of the present invention, should also prevent wear of the dryer surface and provide lubrication between the doctor blade and the dryer surface and reduce chemical corrosion, as well as controlling the extent of creping. A coating that adheres the sheet to the drum will give a good crepe, imparting absorbance and softness with the least possible loss of paper strength. If adhesion to the dryer drum is too strong, the sheet may pick or even “plug”, i.e., underride the doctor blade, and wrap around the dryer drum. If there is not enough adhesion, the sheet will lift off too easily and undergo too little creping. The creping adhesive, as an aqueous solution or dispersion, is usually sprayed onto the surface of the creping cylinder, e.g., a Yankee dryer. This adhesion of the sheet to the Yankee dryer improves heat transfer, allowing more efficient drying of the sheet.

If the web sticks too strongly to the creping cylinder, release agents such as those of the present invention can be sprayed on the cylinder. These release agents aid in the release of the tissue web at the creping blade, lubricate and protect the blade from excessive wear, and modify the properties of the adhesive allowing for control of coating thickness. Known release agents have included materials such as emulsifiable oils, polyphosphates, and various surfactant-type chemistries. Release agents may be added to the wet end, blended with the adhesive and sprayed on the dryer using a single application system, or sprayed separately from the adhesive via a dedicated application system. The mode of action of release agents is such that they intentionally interfere with the formation of the coating. Typically, as the amount of release agent added is increased, the level of adhesion continues to decrease.

Certain hydrophobic chemicals, typically in the form of aqueous emulsions, are applied to paper machine equipment to act as release agents. For example, U.S. Pat. No. 5,658,374 to Glover teaches that an oil-in-water emulsion containing an alcohol, a fatty acid or an oil, and lecithin emulsified with a water-soluble or water-dispersible surfactant can be used to control sticky deposition on the surfaces of press rolls, Yankee rolls and couch rolls surfaces in papermaking In another example, U.S. Pat. No. 5,863,385 to Siebott et. al. teaches a process for cleaning and preventing deposition on paper machine parts, including the press section, by treating the surface with an oil-in-water emulsion. The oil phase can be any of several compounds including saturated hydrocarbons, fatty alcohols, fatty acids, fatty acid esters, paraffin oil, mineral oil or poly-alpha-olefins. In another example, U.S. Pat. No. 6,139,911 to Vanhecke et. al. teaches the use of aqueous microemulsions for improving the release properties of press rolls where the oil phase is selected from oils, water insoluble surfactants, water insoluble polymers, and waxes. The microemulsion is applied by first diluting it with excess water or by applying it directly in the presence of excess water. When the microemulsion is applied to the press roll in either diluted manner, the emulsion breaks up, causing the release components to deposit on the roll surface as larger macroemulsion size (or greater) particles, which are more efficient at affecting release.

U.S. Pat. No. 6,558,513 to Pease et. al. teaches the use of non-curing hydrocarbon polymers, such as polybutene, for improving the release of paper webs from the surface of press rolls or other papermaking equipment or converting equipment. The composition is applied directly to the equipment surface in the absence of water, which would require application to an already cured creping adhesive coating and preclude its use in systems where the adhesive and release agents are blended and sprayed onto the dryer in a single application system. Further, these compositions contain, at minimum, 20% polybutene. U.S. Pat. No. 9,267,239 to Dilkus discloses a method of reducing adhesion to the surface of equipment used in paper manufacture or in paper converting processes, comprising applying to the surface the a composition, comprising (a) 50-98 parts of hydrocarbon oil, (b) 1-40 parts of emulsifier, and (c) 1-10 parts of a non-curing hydrocarbon polymer.

### BRIEF DESCRIPTION OF THE FIGURE

FIG. 1 is a graph that shows lubricity data related to the metal/metal application of Example 1.

### DESCRIPTION OF THE INVENTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, and other published materials referred to throughout the entire disclosure herein, unless noted otherwise, are incorporated by reference in their entirety.

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims, unless the context clearly dictates otherwise. Thus, for example, reference to “a polypeptide” includes one or more of such polypeptides, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

The present invention provides compositions for, and methods of, improving the lubricity or friction reduction between substrates. One aspect of the present invention is a method for improving the lubricity or friction reduction between two metal substrates. Another aspect of the present invention is a method of improving the lubricity of a paper substrate and a metal surface.

The application of lubricity agents or friction reducers are common for all of these industries. Things like PEG esters or fatty alcohols are commonly used. However, there are limitations to what can be used due to their handling properties. Therefore, the industry is often restricted to those that are more water soluble or easily water miscible. These limitations equate to limitations on lubricity improvements or friction reduction. This patent aims to further enhance these properties with a new formulated technology.

One embodiment of the present invention provides a method for improving paper web release from press rolls or other paper processing equipment by interfering with adhesive coating formation, providing additional lubrication between the doctor blade and the dryer surface, and altering the properties of the adhesive allowing for control of coating thickness and degree of adhesion. With respect to paper release, the inventors have found that, surprisingly, the compositions of the present invention show excellent release efficiency

Another embodiment of the present invention is a method of reducing adhesion to a metal-metal surface, or the surface of equipment used in paper manufacture or in paper converting processes, comprising applying to the surface a PEG composition of the present invention, an emulsified PEG, or a composition comprising a PEG.

The method may comprise applying to surface a composition comprised applying to a surface an effective lubricity enhancing amount of at least one PEG in combination with an emulsifying agent and a carrier. One embodiment of the present invention includes methods of improving the lubricity or friction reduction between substrates. The methods include improving the lubricity or friction reduction with emulsified PEG type chemistries.

The compositions can be applied by any means, such as, blending with the adhesive and spraying on the surface using a single application system, or spraying separately from the adhesive via a dedicated application system.

Another embodiment of the present invention provides a method for improving paper web release from press rolls or other paper processing equipment by interfering with adhesive coating formation, providing additional lubrication between the doctor blade and the dryer surface, and altering the properties of the adhesive allowing for control of coating thickness and degree of adhesion. With respect to paper release, the inventors have found that, surprisingly, the compositions of the present invention show excellent release efficiency.

In embodiments of the present invention, the PEG is of the following formula:

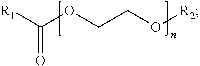

wherein:
R₁ is H, alkyl, stearyl, oleyl; and
R₂ is H, alkyl, stearyl, oleyl.

One method comprises applying to surface a composition comprised of an emulsified C₁₆-C₂₂ diester of PEG. Another aspect is the application of a C₁₆-C₂₂ monoester of PEG. Another aspect is the application of a combination of the di and monoester. In embodiment, the combination is about 90% or higher monoester.

In embodiments of the invention, the emulsified compound is based on the following formula:

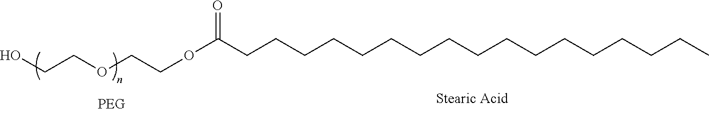

where n is 1-30. Embodiments in the invention include the mono and di stearyl variations. The stearyl containing products are not able to be applied in aqueous systems, as they are insoluble. An emulsion is needed. In embodiments of the invention, the compound is emulsified with sodium oleate.

In one embodiment, the emulsifier is coconut diethanolamide. Thus, one embodiment of the invention is a stearic acid diester of PEG is emulsified with coconut diethanolamide in water.

Examples of the present invention include the PEG compounds shown below:
PEG 400—MonoOleyl ester
PEG 400—DiOleyl
PEG 400—DiOleyl/MonoOleyl
PEG 400—MonoStearyl
PEG 400—DiStearyl
PEG 400—DiStearyl/MonoStearyl
PEG 600—MonoOleyl ester
PEG 600—DiOleyl
PEG 600—DiOleyl/MonoOleyl
PEG 600—MonoStearyl
PEG 600—DiStearyl
PEG 600—DiStearyl/MonoStearyl.

Examples of emulsifiers include, but are not limited to, polyethylene glycol mono- and diesters of fatty acids; linear and branched alcohol ethoxylates; alkyl phenol ethoxylates; phosphate esters of linear and branched alcohol ethoxylates; and quaternary ammonium surfactants. The emulsifiers are present to reduce the interfacial tension at the oil-water interface and stabilize emulsions of the composition prior to and at the point of application.

Other examples of emulsifiers include fatty amides, coconut diethanolamide, and metal salts of a fatty acid. In embodiments, the metal salt of a fatty acid is sodium oleate.

Other examples of emulsifiers include for example, sorbitan fatty acid esters, such as sorbitan monooleate or sorbitan monolaurate, polyoxy ethylene sorbitan fatty acid esters such as fatty acid esters and laurate esters, alkoxylated alcohols, other fatty acids, alkoxylated fatty acids, alkoxylated alkylphenols, sulfates and sulfonates of oils and fatty acids, sucrose and glucose esters and derivatives thereof, as well as aliphatic esters, ethoxylated aliphatic esters and glycerol esters and the like. In some embodiments, water-soluble or water-dispersible surfactants are nonionic surfactants, for example, sorbitan fatty acid esters, polyoxyethylene sorbitan fatty acid esters, alkoxylated alcohols and the like may be used. In certain embodiments, the polyoxyethylene sorbitan fatty acid esters can be Tween 20, Tween 40, Tween 60 and Tween 80, while the sorbitan fatty acid esters can be Span 20, Span 40, Span 60 and Span 80.

Examples of the aqueous base fluid, or carrier, is water or a water/alcohol combination.

The PEG lubricating agent can be present in the lubricating agent/friction reducer composition in an amount ranging from about 0.1% to neat (100% by wt.) The emulsifier can be present in an amount ranging from about 1% to about 15% of the PEG weight; also ranging from about 2-5%. The base fluid, or carrier, can be present in an amount ranging from about 0-99% by wt.

The following is presented for exemplary purposes only. The Examples and exemplary formulations are not intended to be construed as limiting the present invention.

### Example 1—Metal/Metal Surfaces

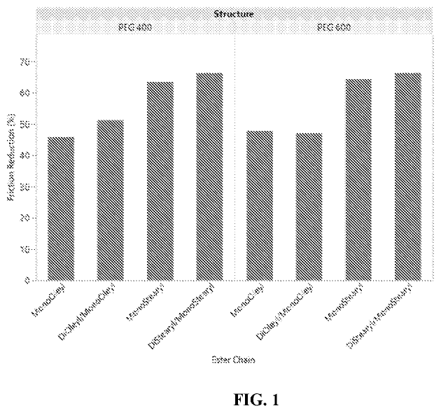

This example demonstrates certain lubricating agents of the present invention. They were evaluated for their ability to improve the lubricity of aqueous systems. The Coefficient of Friction (CoF) & Percent Torque Reduction values for fluids containing the invention of the invention were determined by incorporating the lubricating agent of the current invention in DI water. Standard testing procedures using an OFITE Digital Lubricity Meter (OFI Testing Equipment, Houston, TX) were followed in these studies. This lubricity tester is used to measure the lubricating quality of these lubricity agents, provide data to evaluate the type and quantity of lubricating additives that may be required, and predict wear rates of mechanical parts in known fluid systems. For the standard lubricity coefficient test, 150 in-lb of torque force (the equivalent of 5,000 to 10,000 psi pressure on the intermediate fluid) is applied to two hardened steel surfaces, a block and a ring rotating ring at 60 rpm. Test concentrations were chosen to be 0.6% distearyl PEG, 0.15% coconut diisopropanol amide, and the remainder water. The data in Table 1 was generated using a oleyl and stearyl versions of mono and diester of PEG 400 and 600. Table 1 summarizes both the comparison of coefficient of friction results as well as the friction reduction results for formula. The percent torque reduction is calculated by comparing the difference between the CoF with the lubricating agent and DI water. Also see FIG. 1, showing the lubricity reduction—the difference between the improvement and just water

TABLE 1
Summary of Coefficient of Friction data and Friction Reduction
data for the lubricity agent measured at 72 °F for 5 minutes

| | | | | | Friction |
|---|---|---|---|---|---|
| PEG | | | Lubricity | Coefficient | Reduction |
| Structure | Chain Length | Emulsifier | Measurement | of Lubricity | (%) |
| | | | 35.7 | | 0.0 |
| PEG 400 | DiOleyl/MonoOleyl | None | 17.4 | 0.166 | 51.3 |
| PEG 600 | DiOleyl/MonoOleyl | None | 18.9 | 0.180 | 47.1 |
| PEG 600 | MonoOleyl | None | 18.6 | 0.177 | 47.9 |
| PEG 400 | MonoOleyl | None | 19.3 | 0.184 | 45.9 |
| PEG 600 | DiStearyl/MonoStearyl | coconut diisopropanol | 12 | 0.114 | 66.4 |
| | | amide | | | |
| PEG 600 | MonoStearyl | coconut diisopropanol | 12.7 | 0.121 | 64.4 |
| | | amide | | | |
| PEG 400 | MonoStearyl | coconut diisopropanol | 13 | 0.124 | 63.6 |
| | | amide | | | |
| PEG 400 | DiStearyl/MonoStearyl | coconut diisopropanol | 12 | 0.114 | 66.4 |
| | | amide | | | |

### Example 2: Hair/Plastic Comb

Another example of the present invention is a hair conditioner may compose an emulsified PEG composition of the present invention. One aspect of this is a composition of lactic acid (about 0.6%), stearamidoproyl dimethylamine (about 2.0%), distearyl PEG (about 6%), sodium benzoate (about 1.0%), water (about qs to 100%).

### Example 3: Paper/Metal Surfaces

Another example of the present invention is a paper release aid, such as those described above in paper creping. One aspect of this is a composition of about 0.6% distearyl PEG, about 0.15% coconut diethanolamide, and the remainder water.

Notwithstanding that the numerical ranges and parameters setting forth the broad scope of the invention are approximations, the numerical values set forth in the experimental sections or the example sections are reported as precisely as possible. Any numerical value, however, inherently contain certain errors necessarily resulting from the standard deviation found in their respective testing measurements. While various embodiments of the present invention have been described above, it should be understood that they have been presented by way of example and not limitation. It will be apparent to persons skilled in the relevant art(s) that various changes in form and detail can be made therein without departing from the spirit and scope of the present invention. In fact, after reading the above description, it will be apparent to one skilled in the relevant art(s) how to implement the invention in alternative embodiments. Thus, the present invention should not be limited by any of the above-described exemplary embodiments.

The compositions, processes, systems, and methods of the present invention are often best practiced by empirically determining the appropriate values of the operating parameters, or by conducting simulations to arrive at best design for a given application. Accordingly, all suitable modifications, combinations, and equivalents should be considered as falling within the spirit and scope of the invention.

We claim:

1. A lubricating agent/friction reducer composition, comprising:
an aqueous base fluid;
at least one polyethylene glycol (PEG) and
an emulsifying agent

2. The composition claim 1, wherein the PEG is a high carbon chain fatty acid ester.

3. The composition claim 1, wherein the high carbon chain fatty acid is a C₁₈-C₂₂ ester, preferably a saturated C₁₈ (Stearic).

4. The composition of claim 1, wherein the PEG is of the following formula: 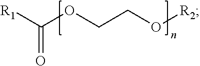
wherein:
R₁ is H, alkyl, stearyl, oleyl; and
R₂ is H, alkyl, stearyl, oleyl.

5. The composition of claim 1, wherein the PEG is of the following formula: 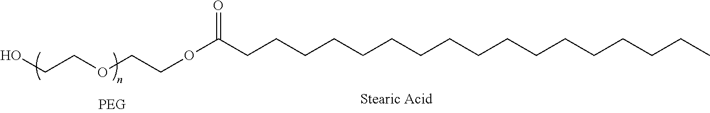
where n is 1-30.

6. The composition of claim 1, wherein the PEG is at least one of: PEG 400—MonoOleyl ester, PEG 400—DiOleyl, PEG 400—DiOleyl/MonoOleyl, PEG 400—MonoStearyl, PEG 400—DiStearyl, PEG 400—DiStearyl/MonoStearyl, PEG 600—MonoOleyl ester, PEG 600—DiOleyl, PEG 600—DiOleyl/MonoOleyl, PEG 600—MonoStearyl, PEG 600—DiStearyl, PEG 600—DiStearyl/MonoStearyl, or a combination thereof.

7. The composition claim 1, wherein the dosage of the ester can be between 0.1% to 99%.

8. The composition claim 1, wherein the emulsifying agent is a fatty amide.

9. The composition claim 1, wherein the emulsifying agent is a metal salt of a fatty acid.

10. The composition claim 1, wherein the metal salt of a fatty acid is sodium oleate.

11. The composition claim 1, wherein the emulsifying agent is coconut diethanolamide.

12. The composition claim 1, wherein the emulsifying agent is coconut diisopropanolamide.

13. The composition claim 1, wherein the dosage of the emulsifying agent can be between 0.01-99%.

14. The composition claim 1, wherein the formula is 0.6% PEG, 0.15% emulsifier, and the remainder water.

15. The composition claim 1, comprising about 0.6% distearyl and/or stearyl PEG, about 0.15% coconut diethanolamide, and the remainder water.

16. A method of improving the lubricity of two substrates, comprising applying at least one emulsified PEG to at least one substrate.

17. A method of improving the lubricity of two substrates, comprising applying a composition of claim 1 to at least one substrate.

18. The method of claim 17, wherein the composition is a composition of one of claims 2-15.

19. A method of reducing adhesion to a surface of equipment used in paper manufacture or in paper converting process comprising the steps of:
a) applying at least one emulsified PEG to the surface;
b) pressing the paper web against the drying surface to adhere the web to the drying surface, and
c) dislodging the web from the drying surface with a creping device to crepe the paper web.

20. The method of claim 19, wherein the PEG is in a composition of one of claims 2-15.
